(19) United States
(12) Patent Application Publication
(10) Pub. No.: US 2007/0196495 A1
(43) Pub. Date: Aug. 23, 2007

(54) Method for administering medicaments to subjects with swallowing difficulties and disorders

(75) Inventor: Richard Soltero, Cary, NC (US)

Correspondence Address: JOHN CULLEY, 2260 AUTUMN WAY, MERIDIAN, ID, 83642, US

(73) Assignee: Richard a. Soltero

(21) Appl. No.: 10/590,282

(22) PCT Filed: Mar. 24, 2005
(86) PCT No.: PCT/US05/09548

§ 371 Date: Aug. 22, 2006

Related U.S. Application Data
(60) Provisional application No. 60/558,349, filed on Mar. 31, 2004.

(51) Int. Cl. A61K 31/714; A61K 31/616; A61K 31/59; A61K 31/56; A61K 31/525; A61K 31/519; A61K 31/51; A61K 31/195; A61K 33/14; A61K 33/26; A61K 33/24

(52) U.S. Cl. 424/488; 514/167; 514/251; 514/276; 514/350; 514/356; 514/455; 514/474; 514/561; 514/725; 514/763; 424/641; 424/688; 424/702; 514/52; 514/165; 424/728; 424/730; 424/752; 424/737; 514/211.07; 514/182; 514/262.1; 514/423; 514/682; 424/646; 424/650; 514/393; 424/655; 424/684; 424/679

(57) ABSTRACT

The present invention is a variably thickened pharmaceutical dosage form, its composition and its use for orally administering medications to patients that have difficulty swallowing other solid dosage forms such as tablets or capsules.

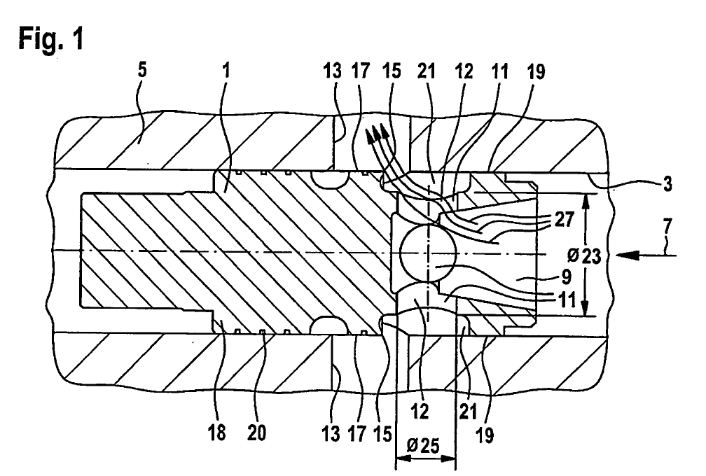

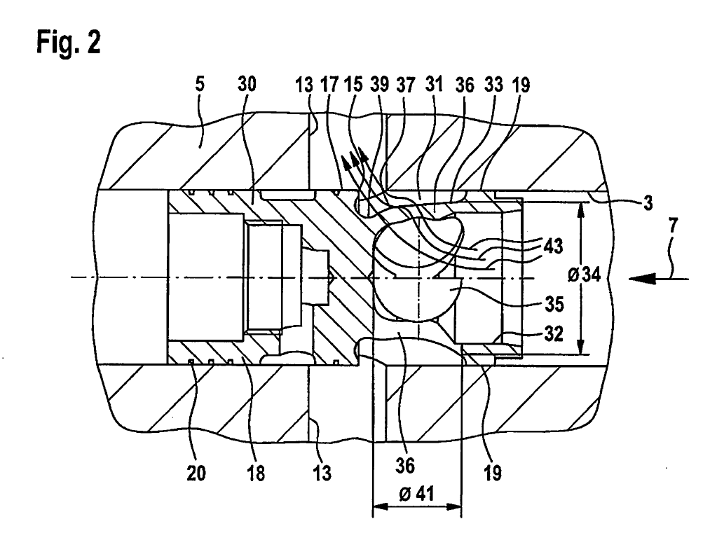

### FIELD OF THE INVENTION

The present invention provides a variably thickened pharmaceutical composition for supplying oral medicaments to a patient demonstrating or at risk for abnormalities in swallowing.

### BACKGROUND OF THE INVENTION

A normal human swallow can be separated into four semi-distinct phases according to Dr. Aviv at the Voice and Swallowing Center, Columbia University. Any one or more of these stages in the swallowing process can become impaired and result in abnormalities in the human swallow, a condition called dysphagia. For example, acute dysphagia may be the result of inflammatory conditions such as pharyngitis, tonsillitis, or aphthous ulceration of the mouth. In addition, a spectrum of very different medical conditions, both physical and neurological in nature, can alter normal swallowing.

A number of approaches are conventionally employed to enable administration of oral medicaments to a subject following a diagnosis of dysphagia or other swallowing disorders. In general, oral solid dosage forms such as tablets, capsules, pills, and powders are not easily taken by a dysphagic patient; and, a liquid or syrup formulation of the prescribed medicament may be substituted, if available. This approach has been described by Dessibourg and Gachoud, for example, as a means for administering the medications levodopa and benserazide in the treatment of patients with Parkinson disease. [C. A. Dessibourg and J. P. Gachoud, Schweiz. Rundsch. Med. Prax. 84(43), 1235-1238, 1995.] Frequently, however, no liquid dosage form of a medicament is commercially available, or the liquid medicament formulation may cause choking, difficulty in swallowing, or regurgitation, or may have an undesirable or bitter taste or after-taste, poor dispensability or instability.

As an alternative, a person providing care to a dysphagic person often attempts to transfer a medicament to a thickened drink or soft food immediately prior to administration. A tablet containing a drug may be partially crushed, for example, and the fragments added to a thickened or viscous liquid or soft food. Likewise, the contents of a capsule may be emptied into a thickened liquid or soft food and dispersed by stirring. Frequently, the fragments of the drug dosage form are not uniformly dispersed, and portions of the original dose remain in the mixing container. Further, the presence of the drug-containing particles in the food or liquid may elicit an abnormal swallowing response, leading to coughing, regurgitation, or aspiration. When this occurs, the net result is a failure to deliver the requisite dose of the medicament to the subject and an enhanced risk of aspiration and its undesirable consequences.

Yet another conventional treatment for patients who have trouble swallowing involves the use of enteral feeding tubes through which a liquid formulation of a drug may be administered. Skilled care-givers must insert the enteral feeding tube. Moreover, use of an enteral feeding tube requires that a liquid formulation of the drug be available and that the drug is compatible with the tube material.

U.S. Pat. No. 6,531,114 teaches methods and delivery vehicles, i.e., chewing gum dosage forms, for delivering a medicament. Chewing creates a pressure within the oral cavity of the individual to force the drug directly into the systemic system of that individual through the oral mucosa of the oral cavity via the buccal or sublingual absorption routes. However, a subject having dysphagia may lack the cognitive skills or oral motor skills to derive benefit from prolonged chewing of chewing gum dosage forms or may suffer coughing, discomfort, choking, and pain by attempting to swallow the chewing gum dosage form.

U.S. Pat. No. 5,932,235 teaches a jellied medical composition for oral administration, which is easily taken by patients of advanced age or patients with dysphagia. U.S. Pat. Nos. 5,558,880 and 5,648,093 teaches a fast dissolving, solid dosage form defined by a matrix containing gelatin, pectin and/or soy fiber protein and one or more amino acids having from about 2 to 12 carbon atoms. The dosage form is formed by subjecting a matrix material solution to lyophilization or solid-state dissolution.

There has been a long-felt and unmet need for a method for the oral administration of medicaments to dysphagic patients and those at risk for swallowing abnormalities, as well as methods for the preparation of compositions that will enable oral administration of medicaments to this population of people. The present invention addresses this need.

### SUMMARY OF THE INVENTION

The present invention provides a solid dosage form that facilitates swallowing comprising a hydrated polymeric gelatinous matrix, one or more active ingredients, and optionally one or more excipients.

The second embodiment of the invention is a method for administering to a patient a solid dosage form that facilitates swallowing comprising a hydrated polymeric matrix, one or more active ingredients, and optionally one or more excipients without water or other fluids needed to facilitate swallowing.

### DETAILED DESCRIPTION

The dosage form of the present invention, because of the gelatinous consistency of its hydrated polymeric matrix, is softly resilient, yet is appropriately firm to facilitate swallowing and passage down the esophagus without hesitation, coughing, pain, and regurgitation. It is cohesive in the mouth, and passes through the throat smoothly when swallowed. Accordingly, it is particularly suitable for medication delivery for patients with dysphagia or other swallowing abnormalities.

The dosage form has ingestion qualities and textural properties allowing it to be readily positioned in the mouth by, e.g., pressing with the tongue, and without chewing smoothly passes through the throat. It stimulates salivation through positive enhancement of taste, smell and/or texture, which further facilitates swallowing.

The essential components in the dosage form are an active ingredient, (i.e. biologically active, therapeutic agent, medicant, plant extract, vitamin, etc.) and a hydrated polymeric material, and one or more secondary ingredients, i.e. excipients, may be optionally added. All non-active ingredient components are food grade or “generally recognized as safe” (GRAS) by those skilled in the art of pharmaceutical preparations, i.e. pharmaceutically acceptable. The dosage form can be made into a variety of shapes including a cylinder wherein its length is greater than diameter, a cylinder with flat ends, a cylinder with tapered ends, a cylinder with one tapered end, and the other end rounded or flat The cross section of the cylinder need not be a true circle, but may be an oval or ellipse. Further, the length and diameter of the cylinder may be approximately equal. The preferred shape is a cylinder wherein its length is greater than its diameter with rounded ends.

The active ingredient(s), alone or in combination with other active ingredients, may include pharmaceuticals agents (over-the-counter, prescription, or new chemical entities (NCE)), vitamins, minerals, and diagnostics or any other biologically active agent or health supplement that is normally administered via swallowing. Examples of pharmaceutical agents that may be incorporated in the gelatinous composition are acetaminophen, captopril, diltiazem, nifedipine, dicyclomine, alprazolam, amitriptyline, clomipramine, propranolol hydrochloride, labetalol, allopurinol, metformin, atenolol, potassium chloride, lithium, levothyroxine sodium, ibuprofen, estrogen, and acetyl salicylic acid. However, substantially any pharmaceutical agent or biologically active agent or combination of biologically active agents may be used as the active ingredient, either by adding the active agent(s) to the mixture to be jellied or by adding solutions, emulsions, liposomes, or complexes of the active agent to the mixture to be jellied. One or more excipients such as preservatives, flavors, antioxidants, surfactants, sweeteners, olfactory inducing agents or colorings may also be incorporated into the formulation.

Hydrateable polymeric materials suitable for preparation of the matrix in the present dosage form include materials derived from animal or vegetable proteins, such as the gelatins, dextrins and soy, wheat and psyllium (see proteins); gums such as acacia, guar, agar, and xanthan; polysaccharides; alginates; carboxymethylcelluloses; carrageenans; dextrans; pectins; synthetic polymers such as polyvinylpyrrolidone and polyacrylic acid polymers such as carboxyvinylpolymers and carbomers; and polypeptide/protein or polysaccharide complexes such as gelatin-acacia complexes. Preferred matrix forming agents include pharmaceutical grade gelatins, pectins (nonhydrolyzed, partially hydrolyzed or hydrolyzed), and hydrolyzed celluloses, either alone or in combination.

Excipients are agents, or other agents that may enhance the physical properties of the composition to aid swallowing or preserve the activity of the active ingredient(s) and optionally may be included alone or in combinations. Example of excipients useful in the present invention include preservatives, olfactory stimulants, salivation stimulants, solubilizing agents, pH modification agents, sweeteners, flavoring agents, antioxidants.

Process for Preparation:

Typically, a hydrateable polymeric matrix material is mixed with water or other appropriate solvent to form a suspension, into which one or more active ingredient(s) and optionally one or more excipients are blended. The mixture is then processed to induce gelling, e.g., heating or cooling depending upon the polymeric matrix. The mixture is then cast into molds wherein it gels. Alternatively, the mixture is allowed to cool and the gel is extruded as the dosage form from the mold. Those knowledgeable in the pharmaceutical arts will recognize that varieties of both natural and synthetic polymers are useful for forming the gelatinous matrix.

Gelatin is graded and sold by its ‘Bloom Value’ that is a measurement of the strength of a gel formed by a 6 and 2/3% solution of the gelatin that has been kept in a constant temperature bath at 10 degrees centigrade (50° F.) for 18 hours. A device called a Texture Analyzer is then used to measure the weight in grams that is required to depress a standard AOAC plunger 4 millimeters into the gel. If this procedure requires 200 grams, then the gelatin is a 200-bloom gelatin. A lower the bloom value produces a weaker gelatin. The three most common grades of gelatin are 125, 175 and 250 although other grades maybe used in this invention.

Other functional characteristics of gelatin can be summarized as follows: natural gelling, thickening, stabilizing, foaming, water binding, whipping, emulsifying and conservation agent. A variety of different textures, hard or soft, short or long, can be obtained by simply changing the concentration and/or Bloom strength of the gelatin. Among the many parameters to consider during the selection process in addition to Bloom Value are firmness, relaxation, swelling, adhesiveness, tack, stickiness, cohesiveness, rupture/burst and extensibility.

Polymeric matracies (gelatin) can have two isoelectric points, depending on the method of preparation. So-called Type A gelatin, derived from an acid-treated precursor, has an isoelectric point of between pH 7 and 9 Type B gelatin, obtained from an alkali-treated precursor, has an isoelectric point of approximately pH 5. Type A gelatin acts best as an emulsifier around pH 3, where it is positively charged. On the other hand, Type B gelatin is best around pH 8, where it is negatively charged. Both Type A and Type B gelatin can be used in this invention. To avoid an incompatibility, all emulsifying agents should carry the same charge.

The gelation temperature or melting point of gelatin-water systems is in the range of 20 to 40° C. The gelation temperature increases with increasing gelatin content and with increasing gelatin molecular weight, as does the solution viscosity. Below the gelation temperature, the gel rigidity increases with increasing gelatin content. While the modulus and the ultimate strength of aqueous gels increase with increasing gelatin content, the elongation at break is not much affected. Gel strength and rigidity are highest at the isoelectric point, where cross-linking by salt bridges is most extensive. While typical aqueous gelatin gels contain 20 to 45% solids (polymeric matrix), at room temperature pectin and agar form strong gels, which contain only 1 to 4% solids. For use in this invention, the percent of polymeric matrix may range from 1 to 75%.

Besides the chemical nature of polymeric matrix and solvent, the three most important factors influencing the gelling of polymer solutions are concentration, temperature, and molecular weight. Lower temperatures, higher concentrations of gelling polymer, and higher molecular weights of gelling materials promote gelling and produce stronger gels.

For a typical gelatin, 10% solutions (solutions containing 10% polymeric matrix) begin to gel at about 25° C.; 20% solutions at about 30° C.; and 30% solutions at about 32° C. With some polymeric matrices, the gelation is reversible; the gels liquefy when heated above these temperatures. Gelation is rarely observed above 34° C. regardless of concentration, so that gelatin solutions do not gel at 37° C. The gelation temperature or gel point is highest at the isoelectric point, where the attachment between different chains by coulombic attraction or ionic bonds between carboxylate groups and alkylammonium, guanidinium or imidazolium groups is most extensive.

The gelation temperature or the melting point of the gel depends more strongly on temperature and concentration than on pH. The combination of an acid pH considerably below the isoelectric point and a temperature of 37° C. completely prevents the gelation of gelatin solutions. Agar and pectic acid solutions set to gels at only a few percent of solids. Unlike most water-soluble polymers, methylcellulose, hydroxypropylcellulose, and polyethylene oxide are more soluble in cold that in hot water. Their solutions therefore tend to gel on heating.

Those skilled in the art will recognize that the dosage form may contain or act as a sustained release formulation. Examples of such dose forms may include microencapsulated, pegylated or other conjugated forms of the active ingredient.

The dosage form of the present invention can include medications to treat a variety of diseases and that those skilled in the art of pharmaceuticals will appreciate that essentially any orally delivered active ingredient is suited for use with this invention.

### EXAMPLES

### Example 1

### Ibuprofen Dosage Form

| | | |
|---|---|---|
| Gelatin | 5 | g |
| Water | 32.5 | ml |
| Ibuprofen | 12.5 | g |

The gelatin is dissolved in the water and the solution is heated at 40-50° C. for 10 minutes. The ibuprofen is mixed with the solution and the mixture is heated for another 5 min. The mixture is then cast into molds and allowed to cool for 5 hours, after which the dosage forms are removed from the molds and packaged.

### Example 2

### Ibuprofen Dosage Form

| | | |
|---|---|---|
| Gelatin | 5 | g |
| Water | 30 | ml |
| Ibuprofen | 30 | g |
| Excpients (flavoring agent, preservative, and anti-oxidant) | 2 | g |

The gelatin is dissolved in the water and the solution is heated at 40-50° C. for 10 minutes. The ibuprofen and excipents are mixed with the solution and the mixture is heated for another 5 min. The mixture is then cast into molds and allowed to cool for 5 hours, after which the dosage forms are removed from the molds and packaged.

### Example 3

### NCE Dosage Form

| | | |
|---|---|---|
| Gelatin | 2 | g |
| Water | 50 | ml |
| Active ingredient | 3 | g |
| Excipents (olfactory agent and preservative) | 5 | g |

The gelatin is dissolved in the water and the solution is heated at 40-50° C. for 10 minutes. The active ingredient may be any pharmaceutical agent amenable to oral administration. The active ingredient and excipents are mixed with the solution and the mixture is heated for another 10 min. The mixture is then cast into molds and allowed to cool for 3 hours, after which the dosage forms are removed from the molds and packaged.

### Example 4

### NCE Dosage Form

| | | |
|---|---|---|
| Gelatin | 2 | g |
| Water | 50 | ml |
| Active ingredient | 20 | g |

The gelatin is dissolved in the water and the solution is heated at 40-50° C. for 10 minutes. The active ingredient is mixed with the solution and the mixture is heated for another 10 min. The mixture is then cast into molds and allowed to cool for 3 hours, after which the dosage forms are removed from the molds and packaged.

I claim:

1. A solid dosage form that facilitates swallowing comprising a gelatinous hydrated polymeric matrix, one or more active ingredients and optionally one or more excipients.

2. The solid dosage form of claim 1 wherein the polymeric matrix is a gel.

3. The dosage form of claim 2 wherein the hydrated gel is hydrated type A gelatin with a bloom value from 0 to 250.

4. The dosage form of claim 2 wherein the hydrated gel is hydrated type B gelatin with a bloom value from 0 to 250.

5. The dosage form of claim 1 wherein the polymeric matrix is an easily hydrated pharmaceutically acceptable polymer.

6. The dosage form of claim 5 wherein the polymeric matrix is hydroxypropyl cellulose.

7. The dosage form of claim 5 wherein the polymeric matrix is hydroxymethyl cellulose.

8. The dosage form of claim 5 wherein the polymeric matrix is polyethylene oxide.

9. The dosage form of claim 5 wherein the polymeric matrix is pectin.

10. The dosage form of claim 5 wherein the polymeric matrix is hyaluronic acid.

11. The dosage form of claim 5 wherein the polymeric matrix is agar.

12. The dosage form of claim 1 wherein an optional excipient is a flavoring agent.

13. The dosage form of claim 1 wherein an optional excipient is a salivation inducing agent.

14. The dosage form of claim 1 wherein an optional excipient is an olfactory agent.

15. The dosage form of claim 1 wherein an optional excipient is a preserving agent.

16. The dosage form of claim 1 wherein an optional excipient is a chemical modifying agent such as pH or solubility.

17. A method of administering an active ingredient to a patient who has a swallowing problem associated with dysphagia comprising administering to the patient a dosage form of claim 1.

18. A method of administering an active ingredient to a pediatric patient who has swallowing difficulties due to physical disorders such as an underdeveloped or small throat comprising administering to the patient a dosage form of claim 1.

19. The dosage form of claim 1 wherein at least one of the active ingredients is a therapeutic chemical, a mineral or vitamin.

20. The dosage form of claim 19 wherein the therapeutic chemical is a mineral or vitamin are selected from the group comprising: ascorbic acid (vitamin C), calcium carbonate, dl-alpha-tocopherol acetate (vitamin E), magnesium oxide, ferrous fumarate, niacinamide, zinc oxide, calcium pantothenate, pyridoxine HCI (vitamin B6), riboflavin (vitamin B2), thiamin mononitrate (vitamin B1), cupric oxide, vitamin A acetate, vitamin D, beta-carotene, chromium chloride, biotin, folic acid, potassium iodide, sodium molybdate, sodium selenate, phytonadione (vitamin K1), sodium metavandate, nickelous sulfate, sodium aluminum silicate, cyanocobalamin (vitamin B12), stannous chloride.

21. The dosage form of claim 1 wherein at least one of the active ingredients is an extract from a plant.

22. The dosage form of claim 21 wherein the plant is selected from the group comprising: echinacea, Ginseng root extract, Ginkgo Biloba, St. Johns Wort.

23. The dosage form of claim 1 wherein at least one of the active ingredients is a non-prescription drug.

24. The dosage form of claim 23 wherein the non-prescription drug is selected from the group comprising: acetaminophen, captopril, diltiazem, nifedipine, dicyclomine, alprazolam, amitriptyline, clomipramine, propranolol hydrochloride, labetalol, allopurinol, metformin, atenolol, potassium chloride, lithium, levothyroxine sodium, Ibuprofen, estrogen, and acetyl salicylic acid.

25. The dosage form of claim 1 wherein at least one of the active ingredients is a prescription drug.

26. The dosage form of claim 25 wherein the prescription drug has indications is selected from the group comprising: Anemia, Anesthesia, Angina, Angioplasty, Antibiotic, Anti-coagulant, Anti-fungal, Arrhythmia, Cancer, Contraceptive, Cystic Crohn's Disease, Fibrosis, Growth hormone deficiency, Hemophilia, Heart attack, Hepatitis, Macular degeneration, Meningococcal meningitis, Multiple Sclerosis, Pulmonary hypertension, Rheumatoid Arthritis and Thrombosis.

26. A variably thickened therapeutic agent composition suitable for oral administration to a subject comprising a uniformly distributed biologically active agent, water and at least one hydrogel-forming component, wherein the composition does not release the biologically active agent in the mouth and that facilitates swallowing.
